(19) United States
(12) Patent Application Publication
(10) Pub. No.: US 2006/0260622 A1
(43) Pub. Date: Nov. 23, 2006

(54) Operating room equipment drapes and methods of making and using the same

(76) Inventors: Deborah M. Wooley, Birmingham, AL (US); Baosheng Lee, Duluth, GA (US)

Correspondence Address: WITHERS & KEYS, LLC, P. O. BOX 2049, MCDONOUGH, GA, 30253, US

(21) Appl. No.: 11/132,579

(22) Filed: May 19, 2005

(51) Int. Cl. A61B 19/00; A61F 5/37

(52) U.S. Cl. 128/849

(57) ABSTRACT

Disposable operating room equipment drapes are disclosed. Methods of making and using disposable operating room equipment drapes are also disclosed.

### FIELD OF THE INVENTION

The present invention relates generally to disposable operating room equipment drapes, methods of making disposable operating room equipment drapes, and methods of using disposable operating room equipment drapes in an operating room setting.

### BACKGROUND OF THE INVENTION

A variety of disposable drapes and linens are used in operating rooms. Disposable drapes and linens may be used to protect and/or cover a patient, to protect and/or cover a piece of operating room equipment, or both. During surgical procedures, it is important for an equipment drape to provide a barrier between the patient and operating room equipment so as to protect the operating room equipment from exposure to body fluids and any other contaminants. Efforts continue in the design of equipment drapes to further enhance the properties of equipment drapes.

What is needed in the art is a disposable operating room equipment drape that (i) provides superior barrier protection to a piece of equipment, (ii) provides a desired absorbency capacity, or (iii) both (i) and (ii).

### SUMMARY OF THE INVENTION

The present invention is directed to a disposable equipment drape suitable for use in an operating room setting. The disposable equipment drape of the present invention provides one or more of the following features: (i) superior barrier protection of a piece of equipment, and (ii) the ability to absorb one or more body fluids from a patient.

According to one exemplary embodiment of the present invention, the disposable operating room equipment drape has an upper surface, a lower surface, and side edges along a periphery of the drape, wherein the equipment drape comprises a layer of liquid pervious nonwoven fabric material extending along the upper surface of the drape; a liquid impervious film layer bonded to at least a portion of the liquid pervious nonwoven fabric material and extending along the lower surface of the drape; and a composite structure positioned between the liquid pervious nonwoven fabric material and the liquid impervious film layer in at least a central portion of the drape, wherein the composite structure comprises (i) a first layer of pulp fibers; (ii) a second layer of pulp fibers; and (iii) a core of cellulosic fibers and superabsorbent material sandwiched between the first and second layers of pulp fibers.

According to a further exemplary embodiment of the present invention, the disposable operating room equipment drape comprises a layer of liquid pervious material extending along an upper surface of the drape; a liquid impervious film layer bonded to at least a portion of the liquid pervious material and extending along a lower surface of the drape; and a composite structure positioned between the liquid pervious material and the liquid impervious film layer in at least a central portion of the drape, wherein the composite structure comprises (i) a first layer of pulp fibers; (ii) a second layer of pulp fibers; and (iii) a core of cellulosic fibers and superabsorbent material sandwiched between the first and second layers of pulp fibers; wherein the drape does not have any fenestrations therein.

The present invention is also directed to an equipment drape assembly. In one exemplary embodiment, the drape assembly comprises (1) an equipment drape comprising (a) a layer of liquid pervious material extending along an upper surface of the drape; (b) a liquid impervious film layer bonded to at least a portion of the liquid pervious material and extending along a lower surface of the drape; and (c) an absorbent composite structure positioned between the liquid pervious material and the liquid impervious film layer in at least a central portion of the drape, wherein the drape does not have any fenestrations therein; and (2) an operating room table optionally having a table mattress thereon, wherein the lower surface of the equipment drape is positioned adjacent an upper surface of the operating room table or the optional table mattress thereon.

The present invention is further directed to methods of preparing an operating room equipment drape. In one exemplary embodiment of the present invention, the method of preparing an operating room equipment drape comprises the steps of providing a layer of liquid pervious nonwoven fabric material; providing a liquid impervious film layer; positioning a composite structure between the liquid pervious nonwoven fabric material and the liquid impervious film layer in at least a central portion of the drape, wherein the composite structure comprises (i) a first layer of pulp fibers; (ii) a second layer of pulp fibers; and (iii) a core of cellulosic fibers and superabsorbent material sandwiched between the first and second layers of pulp fibers; and bonding at least a portion of the liquid pervious nonwoven fabric material to at least a portion of the liquid impervious film layer.

The present invention is even further directed to methods of using an operating room equipment drape in an operating room setting. In one exemplary embodiment of the present invention, the method comprises providing a barrier between a patient and at least a portion of a piece of equipment in an operating room setting, wherein the method comprises the steps of positioning an equipment drape over at least a portion of a piece of equipment. The equipment drape may be any of the above-described equipment drapes of the present invention. In one exemplary embodiment of the present invention, the piece of equipment comprises an operating room table.

These and other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 depicts a top view of an exemplary disposable operating room equipment drape of the present invention;

FIG. 2 depicts a view of the exemplary disposable operating room equipment drape of FIG. 1 on an operating room table;

FIG. 3 depicts a cross-sectional view of the exemplary disposable operating room equipment drape of FIG. 1 along line A-A shown in FIG. 1; and

FIG. 4 depicts a view of a lower surface of an exemplary disposable operating room equipment drape showing an optional cuff feature of the drape.

### DETAILED DESCRIPTION OF THE INVENTION

The present invention is directed to disposable operating room equipment drapes for use in an operating room setting. The present invention is further directed to methods of making and using disposable operating room equipment drapes to properly prepare an operating room setting prior to exposing the setting to a patient. The equipment drapes of the present invention are particularly useful in providing a barrier between a patient and one or more pieces of equipment in an operating room setting.

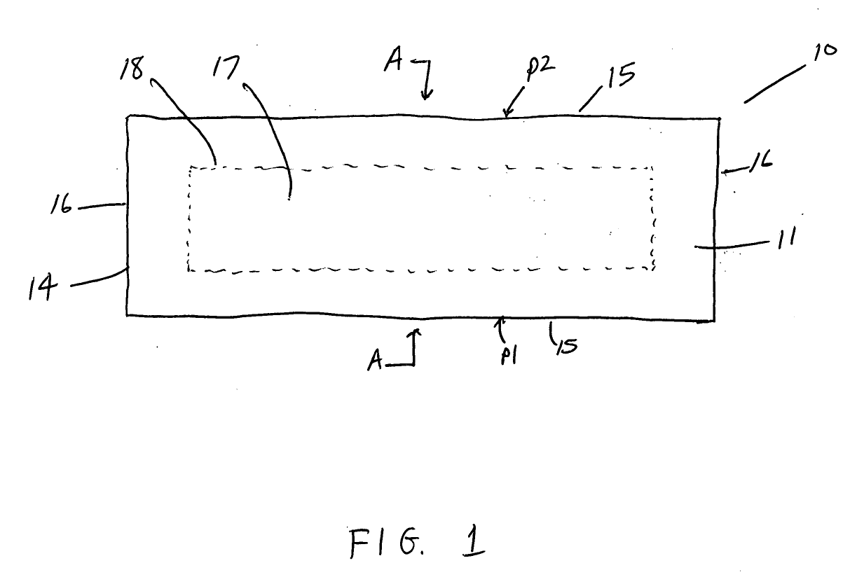

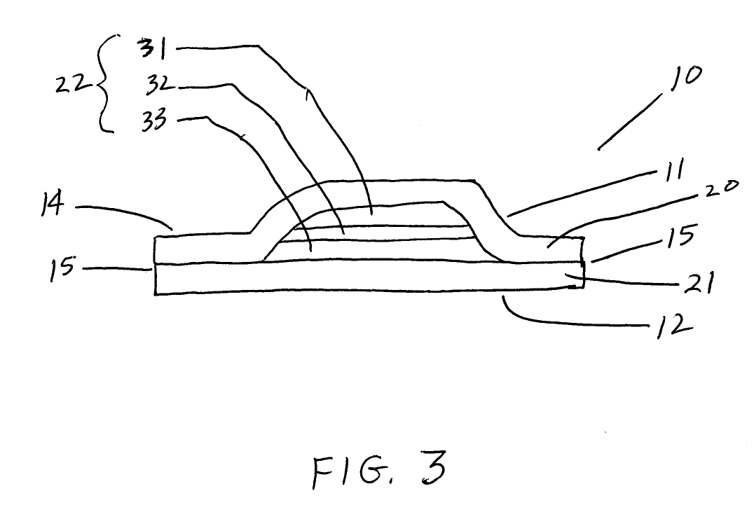

An exemplary disposable operating room equipment drape 10 is shown in FIG. 1. Exemplary disposable operating room equipment drape 10 has an upper surface 11, a lower surface 12 (see FIG. 3) that is suitable for contacting a piece of equipment, such as an operating room table or operating room table mattress, and an outer periphery 14 extending along outer edges 15 and 16 of exemplary disposable operating room equipment drape 10. Area 17 bound by dashed line 18 identifies an area of exemplary disposable operating room equipment drape 10 having enhanced fluid absorbency properties. Although exemplary disposable operating room equipment drape 10 is shown as having a rectangular shape and area 17 is shown as having a rectangular shape, it should be noted that each of exemplary disposable operating room equipment drape 10 and area 17 may have any desired shape independent of one another, and area 17 may extend a complete length and/or width of exemplary disposable operating room equipment drape 10.

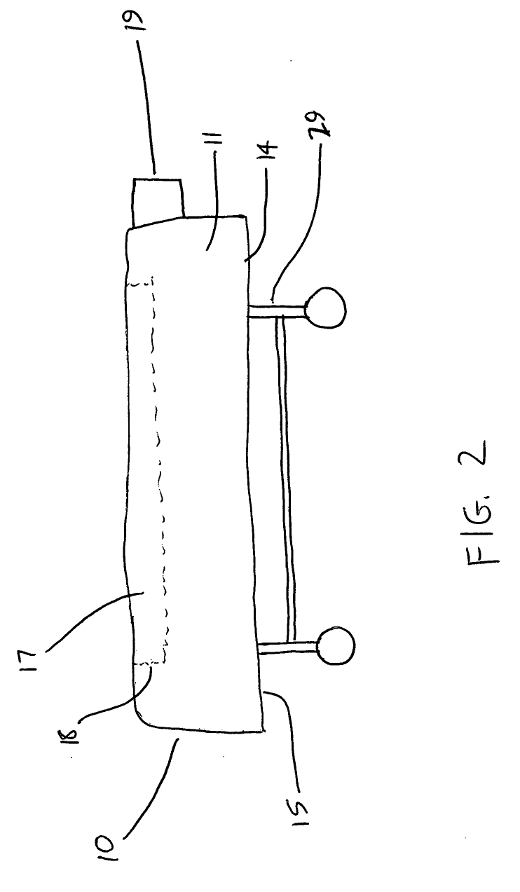

FIG. 2 depicts the use of exemplary disposable operating room equipment drape 10 to cover at least a portion of a piece of equipment, in this particular case, exemplary operating room table 29 having operating room table mattress 19 thereon. As shown in FIG. 2, exemplary disposable operating room equipment drape 10 can cover a portion of exemplary operating room table 29, or alternatively, can cover all of a piece of equipment, such as exemplary operating room table 29.

The operating room equipment drapes of the present invention may comprise at least three distinct layers of material, each of which provides a desired property to the resulting drape. A cross-sectional view of exemplary disposable equipment drape 10 viewed along line A-A of FIG. 1 is provided in FIG. 3. As shown in FIG. 3, exemplary disposable operating room equipment drape 10 comprises a liquid pervious layer 20 forming upper surface 11 of equipment drape 10, a liquid impervious film layer 21 forming lower surface 12 of drape 10, and a composite structure 22 positioned between liquid pervious layer 20 and liquid impervious film layer 21. As described below, composite structure 22 may comprise a number of individual layers, such as layers 31, 32 and 33 shown in FIG. 3.

In the exemplary embodiment of the present invention shown in FIG. 3, composite structure 22 is positioned in a central portion of equipment drape 10 between liquid pervious layer 20 and liquid impervious film layer 21 such that at least a portion of liquid pervious layer 20 is bonded to at least a portion of liquid impervious film layer 21 along outer periphery 14. It should be noted that other configurations are encompassed by the present invention wherein composite structure 22 is positioned in any location between liquid pervious layer 20 and liquid impervious film layer 21. Further, composite structure 22 may extend over a small portion of the overall surface area of exemplary disposable equipment drape 10 or may extend over the entire overall surface area of exemplary disposable equipment drape 10 between liquid pervious layer 20 and liquid impervious film layer 21.

As described above, the disposable operating room equipment drapes of the present invention may comprise a number of components. A description of some of the suitable exemplary components is provided below.

I. Disposable Operating Room Equipment Drapes

The disposable operating room equipment drapes of the present invention may comprise, but are not limited to, one or more of the following components.

A. Operating Room Equipment Drape Components

The disposable operating room equipment drapes of the present invention may comprise one or more of the following components.

1. Liquid Pervious Layer

The disposable operating room equipment drapes of the present invention comprise a liquid pervious layer such as exemplary liquid pervious layer 20 of exemplary disposable operating room equipment drape 10 shown in FIG. 3. The liquid pervious layer enables fluids coming into contact with an upper surface of the equipment drape to enter into the equipment drape and away from the upper surface of the drape.

The liquid pervious layer of the disposable operating room equipment drapes may comprise one or more layers of liquid pervious material positioned adjacent to and/or bonded to one another. Suitable layers include, but are not limited to, perforated film layers; fabric layers such as woven, nonwoven and knitted fabric layers; foam layers; and combinations thereof. In one exemplary embodiment, the liquid pervious layer comprises a nonwoven fabric, such as a spunbonded fabric, a spunlaced fabric, a needle-punched fabric, or any combination thereof. In one desired embodiment, the liquid pervious layer comprises a nonwoven fabric, such as a spunbonded fabric.

Each of the liquid pervious layers may be formed from a variety of materials. Suitable materials include, but are not limited to, cellulosic materials (e.g., pulp fibers), polypropylene, polybutylene, polyethylene terephthalate, polyamide, and combinations thereof. Desirably, the material is hydrophilic with the material either being inherently hydrophilic or rendered hydrophilic due to some modification of the material (e.g., the incorporation of an additive into or onto the material). In one exemplary embodiment, the material comprise polypropylene, such as polypropylene fibers alone or in combination with cellulosic fibers (e.g., pulp fibers). Commercially available polypropylenes suitable for use in the present invention include, but are not limited to, polypropylene available from Basell Polyolefins (Wilmington, Del.) under the trade designation Basell.

When the liquid pervious layer comprises a nonwoven fabric layer, the fibers of the nonwoven fabric layer desirably have an average fiber diameter of less than about 100 microns. More desirably, the fibers of the nonwoven fabric layer have an average fiber diameter of from about 0.5 micron to about 40 microns. Even more desirably, the fibers have an average fiber diameter of from about 1 micron to about 30 microns.

The liquid pervious layer desirably has an overall basis weight (i.e., a basis weight of the one or more liquid pervious layers combined) of less than about 1000 grams per square meter (gsm). More desirably, the liquid pervious layer has an overall basis weight of from about 25 gsm to about 500 gsm. Even more desirably, the liquid pervious layer has an overall basis weight of from about 30 gsm to about 100 gsm.

The liquid pervious layer may have an overall thickness (i.e., a thickness of the one or more liquid pervious layers combined), which varies depending upon the particular end use of the drape. Desirably, the liquid pervious layer has an overall thickness of less than about 1000 microns (μm). More desirably, the liquid pervious layer has an overall thickness of from about 10 μm to about 500 μm. Even more desirably, the liquid pervious layer has an overall thickness of from about 20 μm to about 100 μm.

In one exemplary embodiment, the liquid pervious layer comprises a spunbonded fabric of hydrophilic polypropylene fibers, wherein the spunbonded fabric has a basis weight of about 22 gsm (0.65 oz/yd²). Suitable polypropylene spunbonded fabrics are commercially available from a number of manufacturers including, but not limited to, BBA Fiberweb™ Simpsonville, Inc. (Simpsonville, S.C.).

2. Liquid Impervious Layer

The disposable operating room equipment drapes of the present invention further comprise a liquid impervious layer such as exemplary liquid impervious layer 21 of exemplary disposable operating room equipment drape 10 shown in FIG. 3. The liquid impervious layer of the disposable operating room equipment drapes may comprise one or more layers of liquid impervious material positioned adjacent to and/or bonded to one another.

Each of the liquid impervious layers may comprise any polymeric film-forming material. Suitable film-forming materials include, but are not limited to, polypropylene, polypropylene, polybutylene, polyethylene terephthalate, polyamide, and combinations thereof. Desirably, the polymeric film-forming material comprises a material having a relatively high coefficient of friction or degree of tackiness so that the equipment drape does not slip off of a piece of equipment, such as an operating room table, during use. One desired polymeric film-forming material having a relatively high coefficient of friction comprises polyethylene. Commercially available polyethylenes having a relatively high coefficient of friction that are suitable for use in the present invention include, but are not limited to, polyethylenes available from Winpak, Ltd. (Minneapolis, Minn.) under the trade designation F15001 (e.g., COF greater than 0.4 using ASTM D1894).

As used herein, the term “relatively high coefficient of friction” is used to describe a coefficient of friction of at least 0.2, desirably, greater than 0.4 as measured by ASTM D1894 using an Instron Universal Tester commercially available from Instron Corporation (Norwood, Mass.).

The liquid impervious layer may have an overall thickness (i.e., a thickness of the one or more liquid impervious layers combined), which varies depending upon the particular end use of the equipment drape. Desirably, the liquid impervious layer has an overall thickness of less than about 100 microns (μm). More desirably, the liquid impervious layer has an overall thickness of from about 10 μm to about 50 μm. Even more desirably, the liquid impervious layer has an overall thickness of from about 10 μm to about 40 μm. In one exemplary embodiment, the liquid impervious layer comprises a polyethylene film having an overall thickness of about 38 μm (1.5 mil), and a COF of at least 0.2, desirably, greater than 0.4 as measured by ASTM D1894 using an Instron Universal Tester commercially available from Instron Corporation (Norwood, Mass.).

In some embodiments of the present invention, at least a portion of the liquid impervious layer is bonded to at least a portion of the above-described liquid pervious layer. In one exemplary embodiment, such as exemplary disposable operating room equipment drape 10 shown in FIGS. 1-3, the liquid impervious layer is bonded to the liquid pervious layer along an outer periphery of the drape. In other embodiments (not shown), the liquid impervious layer is bonded to the liquid pervious layer along one or more edges of the drape, such as along opposite edges 15 shown in FIG. 1 or along opposite edges 16 shown in FIG. 1.

3. Absorbent Composite Structure

The disposable operating room drapes of the present invention also comprise an absorbent composite structure such as exemplary composite structure 22 of exemplary disposable operating room equipment drape 10 shown in FIG. 3.

The absorbent composite structure comprises superabsorbent particles (SAP) in combination with pulp fibers. The absorbent composite structure may comprise one or more layers, wherein at least one layer comprises superabsorbent particles (SAP) in combination with pulp fibers. Each SAP-containing layer may comprise a substantially uniform distribution of superabsorbent particles within a given layer, or alternatively, may comprise a non-uniform distribution of superabsorbent particles within a given layer. For example, as described above, the equipment drape may comprise an absorbent composite structure comprising a layer of superabsorbent particles within a central location of the drape (e.g., an area positioned directly above a central location of an operating room table and/or mattress), but be substantially free of superabsorbent material or particles in an outer periphery of the drape.

Any known superabsorbent material may be used in the drapes of the present invention. Suitable commercially available superabsorbent materials include, but are not limited to, superabsorbent particles commercially available from Stockhausen (Greensboro, N.C.) and Dow Chemical (Midland, Mich.).

In one exemplary embodiment of the present invention, the disposable operating room equipment drape comprises at least three separate layers in combination with one another: (1) a liquid pervious layer formed from a nonwoven fabric material, such as a spunbonded polypropylene fabric layer, as the uppermost layer; (2) a liquid impervious polyethylene film layer bonded to at least a portion of the liquid pervious layer, wherein the polyethylene film has a relatively high coefficient of friction; and (3) a superabsorbent layer positioned between the liquid pervious layer and the liquid impervious film layer in a central portion of the equipment drape, wherein the superabsorbent layer comprises a composite structure produced and provided by Rayonier, Inc. (Jesup, Ga.) (hereinafter, “Rayonier”). Absorbent composite structures suitable for use in the present invention include those commercially available from Rayonier and disclosed in one or more of the following U.S. patents assigned to Rayonier: U.S. Pat. Nos. 6,485,667; 5,916,670; and 5,866,242. Commercially available absorbent composite structures suitable for use in the present invention include, but are not limited to, NOVATHIN® absorbent products available from Rayonier.

In a further exemplary embodiment of the present invention shown in FIG. 3, an exemplary disposable operating room equipment drape 10 comprises at least three separate layers in combination with one another: (1) liquid pervious layer 20 desirably formed from a nonwoven fabric material, such as a spunbonded polypropylene fabric layer, as the uppermost layer; (2) liquid impervious film layer 21 bonded to at least a portion of liquid pervious layer 21 along outer periphery 14, wherein liquid impervious film layer desirably comprises a polyethylene film having a relatively high coefficient of friction; and (3) absorbent composite structure 22 positioned between liquid pervious nonwoven fabric material 20 and liquid impervious film layer 21 in at least a central portion of drape 10, wherein absorbent composite structure 22 comprises (i) a first layer of pulp fibers 31; (ii) a second layer of pulp fibers 33; and (iii) a core of cellulosic fibers and superabsorbent material 32 sandwiched between first layer of pulp fibers 31 and second layer of pulp fibers 33.

The pulp fibers within absorbent composite structure 22 desirably have an average fiber diameter of less than about 100 microns. More desirably, the pulp fibers of the absorbent composite structure have an average fiber diameter of from about 0.5 micron to about 40 microns. Even more desirably, the pulp fibers have an average fiber diameter of from about 1 micron to about 30 microns.

The absorbent composite structure desirably has an overall basis weight (i.e., a basis weight of the one or more pulp fiber layers and SAP-containing layers combined) of less than about 1000 grams per square meter (gsm). More desirably, the absorbent composite structure has an overall basis weight of from about 25 gsm to about 500 gsm. Even more desirably, the absorbent composite structure has an overall basis weight of from about 30 gsm to about 100 gsm.

The absorbent composite structure may have an overall thickness (i.e., a thickness of the one or more pulp fiber layers and SAP-containing layers combined), which varies depending upon the particular end use of the drape. Desirably, the absorbent composite structure has an overall thickness of less than about 1000 microns (μm). More desirably, the absorbent composite structure has an overall thickness of from about 10 μm to about 500 μm. Even more desirably, the absorbent composite structure has an overall thickness of from about 20 μm to about 100 μm.

4. Additives

Any of the above-described disposable operating room equipment drape components of the present invention may further comprise one or more additives coated onto or incorporated into one or more of the materials used to form the individual components. Suitable additives include, but are not limited to, antimicrobial agents, colorants, additives to increase the coefficient of friction of a given component layer, additives to increase the hydrophilicity of a given component layer, etc. In one desired embodiment of the present invention, one or more components of the disposable operating room equipment drape comprise an antimicrobial agent incorporated therein. Suitable antimicrobial agents include, but are not limited to, triclosan and other antimicrobial agents commercially available under the trade designation MICROBAN® from Microban International, Ltd. (New York, N.Y.).

For example, one or more of liquid pervious layer 20, liquid impervious film layer 21, and absorbent composite structure 22 may contain one or more of the above-mentioned additives, such as antimicrobial agents commercially available under the trade designation MICROBAN®.

The various additives may be added to a polymer melt and extruded to incorporate the additive into a fiber or film component. Alternatively, one or more additives may be coated onto a fiber or film during or after the fabric or film forming process. Typically, when present, each of the one or more additives is present in an amount less than about 25 weight percent, desirably, up to about 2.5 percent, based on the total weight of the fiber, film or fabric.

B. Other Operating Room Equipment Drape Features

In addition to the above-described components, the disposable operating room equipment drapes of the present invention may comprise one or more of the following features.

1. Dimensions

Operating room equipment drapes of the present invention have dimensions sufficient to cover at least a portion of a piece of equipment, and in some embodiments, the entire piece of equipment. Exemplary pieces of equipment include, but are not limited to, an operating room table, an operating room table mattress, a cart, etc. Typically, the operating room equipment drape has dimensions so that the operating room equipment drape completely covers an upper surface of an object, such as an operating room table or mattress; however, it should be understood that there is no limitation whatsoever regarding the dimensions of the operating room equipment drapes of the present invention.

In one exemplary embodiment of the present invention, the operating room equipment drape has a width ranging from about 24 to about 60 inches, and a length ranging from about 48 to about 104 inches. For example, the operating room equipment drape may have a width of about 40 inches, and a length of about 96 inches, with an absorbent composite structure occupying a central portion of the equipment drape and having a width of 20 inches and a length of 72 inches. In another example, the operating room equipment drape may have a width of about 40 inches, and a length of about 96 inches, with an absorbent composite structure occupying a central portion of the equipment drape and having a width of 20 inches and a length of 96 inches.

2. Elastic Hemline

In a further embodiment of the present invention, the operating room equipment drape may comprise an elastic hemline along at least a portion of the outer periphery. For example, as shown in FIG. 1, exemplary operating room equipment drape 10 can have an elastic hemline extending from point P1 along outer edges 15 and 16 to point P2. In other embodiments, the elastic hemline may extend around the entire outer periphery of the operating room equipment drape. The presence of an elastic hemline enables the disposable operating room equipment drape of the present invention to better fit onto an operating table or table mattress similar to the way an elastic hemline in a bedsheet enables the bedsheet to better fit onto a bed mattress.

3. Cuff

In a further embodiment of the present invention, the operating room equipment drape may comprise a cuff on a lower surface of the liquid impervious layer. The cuff forms a “pocket” on the lower side of the equipment drape. The cuff or pocket is sized so as to fit over at least a portion of an operating room table mattress positioned on an operating room table, or an operating room table mattress in combination with an operating room table. The cuff may be any size such that the cuff fits over at least a portion of any intended substrate or piece of equipment.

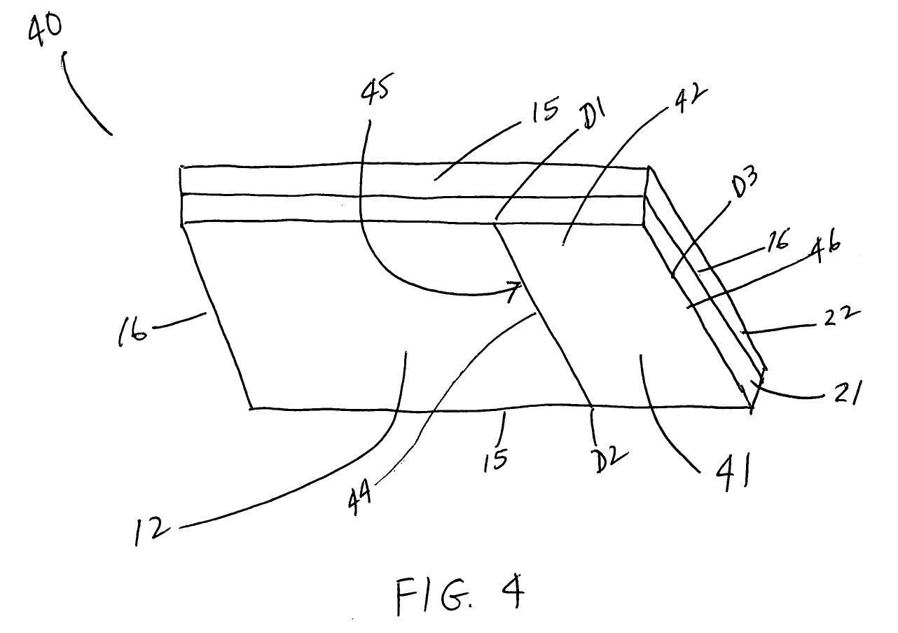

In an exemplary embodiment of the present invention shown in FIG. 4, exemplary operating room equipment drape 40 comprises cuff 41 extending along lower surface 12 of equipment drape 40. Cuff 41 is desirably formed by joining cuff material 42 to liquid impervious layer 21 along outer edges 15 and 16 between points D1 and D2 through point D3. Cuff 41 has a closed end 46 and an open end 44 extending across a width of exemplary disposable operating room equipment drape 40 from point D1 to point D2. Open end 44 of cuff 41 may be separated from liquid impervious layer 21 to access pocket 45 between cuff material 42 and liquid impervious layer 21. Cuff 41 and pocket 45 contained therein are desirably sized so as to fit over at least a portion of an operating room table having an opening therein, a table mattress having an opening therein, or both.

In one exemplary embodiment, cuff 41 extends across a width of liquid impervious layer 21, and along a length of liquid impervious layer 21 from closed end 46 of cuff 41 at a first end of liquid impervious layer 21 to an open end 44 of cuff 41 at a point between closed end 46 and opposite edge 16 of drape 40. Typically, cuff 41 and open end 44 of cuff 41 extend across the full width of liquid impervious layer 21. Further, cuff 41 typically extends about ¼ to about ¾ of the length of drape 40.

The cuff feature of the operating room equipment drape enables a single person to quickly and properly cover an operating room table and/or table mattress. In an exemplary method, a person secures the cuff of the equipment drape over the mattress to stabilize the sheet or drape. Then, the person pulls the disposable operating room equipment drape from head to foot to cover the operating room table and/or table mattress. When the operating room equipment drape further comprises an elastic hemline as described above, the operating room equipment drape including a cuff and an elastic hemline fits snugly around an operating room table and/or table mattress.

II. Methods of Making Disposable Operating Room Equipment Drapes

The present invention is further directed to methods of making an operating room equipment drape. Any of the above-described individual components used to form the operating room equipment drapes of the present invention may be formed using conventional methods. For example, liquid impervious film layers may be forming via any film-forming process including, but not limited to, a film extrusion process, a film-blowing process, etc. Fiber-containing layers, such as a nonwoven fabric layer, may be formed using conventional web-forming processes including, but not limited to, meltblowing processes, spunbonding processes, spunlacing processes, hydroentangling processes, carding processes, needlepunching processes, etc.

Films and fabric layers may be joined to one another using any conventional bonding technique including, but not limited to, thermal bonding processes, adhesive bonding, etc. In one exemplary embodiment of the present invention, a liquid impervious layer may be bonded to a nonwoven fabric layer using a conventional point-bonding apparatus, wherein thermal bonds are used to join the liquid impervious layer to the nonwoven fabric layer. The degree of bonding, size of individual point bonds, and concentration of point bonds may vary as desired.

The absorbent component may be positioned along an outer surface of the liquid impervious layer or the liquid pervious layer prior to joining a portion of the liquid impervious layer to the liquid pervious layer. The absorbent component may simply rest on an outer surface of the liquid impervious layer or the liquid pervious layer (i.e., not bonded to the outer surface), be temporarily attached to the outer surface of the liquid impervious layer or the liquid pervious layer using an adhesive or any other method of temporarily bonding one layer to another (e.g., using a bond that may be easily undone and repositioned if so desired), or be more permanently attached to the outer surface of the liquid impervious layer or the liquid pervious layer using an adhesive or any other method of permanently bonding one layer to another (e.g., using a bond that is not easily undone without damage to one or more layers).

When a cuff is present, the cuff material is typically formed from the same material as the liquid impervious layer. A separate sheet of cuff material may be bonded to a lower surface of the liquid impervious layer using conventional bonding techniques described above. Alternatively, the cuff material and the liquid impervious layer may be a continuous piece of material that is cut an then bonded to itselfto form the cuff using conventional bonding techniques described above.

III. Methods of Using Disposable Operating Room Equipment Drapes

The present invention is further directed to methods of using the above-described operating room equipment drapes in an operating room setting. In one exemplary embodiment, the method comprises a method of providing a barrier between a patient and a piece of equipment in an operating room setting, wherein the method comprises the step of positioning the equipment drape over at least a portion of the piece of equipment to separate the patient from at least a portion of the piece of equipment. Typically, the equipment drape is used to cover the entire piece of equipment. In one desired embodiment, the piece of equipment comprises an operating room table and/or operating room table mattress.

In some embodiments, the method may further comprise one or more of the following steps: removing the equipment drape from a packaging material, opening a cuff on the equipment drape and extending the cuff over at least a portion of a piece of equipment (e.g., an operating room table and/or operating room table mattress), and tucking a portion of the equipment drape underneath at least a portion of a piece of equipment (e.g., an operating room table and/or operating room table mattress).

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A method of providing a barrier between a patient and a piece of equipment in an operating room setting, said method comprising the steps of:
positioning an equipment drape over at least a portion of the piece of equipment to separate the patient from the portion of the piece of equipment, wherein the equipment drape comprises:
a layer of liquid pervious material extending along an upper surface of the drape;
a liquid impervious film layer bonded to at least a portion of the liquid pervious material and extending along a lower surface of the drape; and
a composite structure positioned between the liquid pervious material and the liquid impervious film layer in at least a central portion of the drape;
wherein the equipment drape does not have any fenestrations therein.

2. The method of claim 1, wherein the composite structure comprises:
a first layer of pulp fibers;
a second layer of pulp fibers; and
a core of cellulosic fibers and superabsorbent material sandwiched between the first and second layers of pulp fibers

3. The method of claim 1, wherein the liquid pervious material comprises a spunbonded polypropylene fabric layer, and the liquid impervious film layer comprises a polyethylene film, said polyethylene film having an outer surface that has a coefficient of friction of at least about 0.2 as measured by ASTM D1894.

4. The method of claim 2, wherein the first and second layers are essentially free of superabsorbent material.

5. The method of claim 1, wherein the piece of equipment comprises an operating room table.

6. The method of claim 1, further comprising:
extending a cuff of the equipment drape over a portion of the piece of equipment so that the portion of the piece of equipment is within a pocket of the cuff.

7. An equipment drape having an upper surface, a lower surface, and side edges along a periphery of the drape, wherein the equipment drape comprises:
a layer of liquid pervious material extending along an upper surface of the drape;
a liquid impervious film layer bonded to at least a portion of the liquid pervious material and extending along a lower surface of the drape; and
a composite structure positioned between the liquid pervious material and the liquid impervious film layer in at least a central portion of the drape;
wherein the drape does not have any fenestrations therein.

8. The equipment drape of claim 7, wherein the composite structure comprises:
a first layer of pulp fibers;
a second layer of pulp fibers; and
a core of cellulosic fibers and superabsorbent material sandwiched between the first and second layers of pulp fibers.

9. The equipment drape of claim 7, wherein the liquid pervious material comprises a spunbonded polypropylene fabric layer, and the liquid impervious film layer comprises a polyethylene film, said polyethylene film having an outer surface that has a coefficient of friction of at least about 0.2 as measured by ASTM D1894.

10. The equipment drape of claim 9, wherein the outer surface of the polyethylene film has a coefficient of friction of at least about 0.4 as measured by ASTM D1894.

11. The equipment drape of claim 8, wherein the first and second layers are essentially free of superabsorbent material.

12. The equipment drape of claim 7, wherein the absorbent composite structure extends a length of the equipment drape.

13. The equipment drape of claim 7 in combination with an operating room table, wherein the lower surface of the equipment drape is adjacent an upper surface of (i) the operating room table or (ii) an operating room table mattress on the operating room table.

14. The equipment drape of claim 7, further comprising a cuff on a lower surface of the liquid impervious film layer, wherein the cuff is sized so as to fit over a portion of a piece of equipment.

15. The equipment drape of claim 7, further comprising an elastic hemline extending along at least a portion of the periphery of the drape.

16. A drape assembly comprising:
an equipment drape comprising:
a layer of liquid pervious material extending along an upper surface of the drape;
a liquid impervious film layer bonded to at least a portion of the liquid pervious material and extending along a lower surface of the drape; and
an absorbent composite structure positioned between the liquid pervious material and the liquid impervious film layer in at least a central portion of the drape,
wherein the equipment drape does not have any fenestrations therein; and
an operating room table optionally having a table mattress thereon, wherein the lower surface of the equipment drape is positioned adjacent (i) an upper surface of the operating room table or (ii) an upper surface of the optional table mattress.

17. The drape assembly of claim 16, wherein the absorbent composite structure of the equipment drape comprises:
a first layer of pulp fibers;
a second layer of pulp fibers; and
a core of cellulosic fibers and superabsorbent material sandwiched between the first and second layers of pulp fibers

18. The drape assembly of claim 16, wherein the liquid pervious material of the equipment drape comprises a spunbonded polypropylene fabric layer, and the liquid impervious film layer of the equipment drape comprises a polyethylene film, said polyethylene film having an outer surface that has a coefficient of friction of at least about 0.2 as measured by ASTM D1894.

19. The drape assembly of claim 16, wherein the equipment drape further comprises a cuff on a lower surface of the liquid impervious film layer, wherein the cuff is sized so as to fit over a portion of (i) the operating room table, (ii) the table mattress, or (iii) both (i) and (ii).

20. The drape assembly of claim 16, wherein the equipment drape further comprises an elastic hemline extending along at least a portion of the periphery of the equipment drape.
